(19) United States
(12) Patent Application Publication
(10) Pub. No.: US 2002/0154803 A1
(43) Pub. Date: Oct. 24, 2002

(54) Biological sample carrier

(75) Inventors: Shlomo Berliner, Givatayim (IL); Benny Ron, Savyon (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Inflamet Ltd.

(21) Appl. No.: 10/140,878

(22) Filed: May 9, 2002

Related U.S. Application Data
(63) Continuation-in-part of application No. 09/818,855, filed on Mar. 28, 2001.

(30) Foreign Application Priority Data
Nov. 1, 1999 (IL) 132687
Oct. 23, 2000 (IL) PCT/IL00/00673

(51) Int. Cl. G06K 9/00

(52) U.S. Cl. 382/133

(57) ABSTRACT

A fluid sample carrier is provided. The fluid sample carrier includes at least one lane occupying a length, and a portion of a width, of a surface of the carrier. The lane is sized and configured such that a fluid sample deposited thereupon would flow within the lane upon exertion of a directional force upon the fluid sample. The fluid sample carrier is also designed to prevent contamination of the apparatus in which it is held.

This Application is a Continuation-In-Part of U.S. patent application Ser. No. 09/818,855, filed Mar. 28, 2001, the specification of which is hereby incorporated by reference.

### FIELD AND BACKGROUND OF THE INVENTION

The present invention relates to the field of specimen analysis and, more particularly, to a sample carrier designed to hold and display a fluid sample, such as a biological fluid sample.

Blood analysis and the examination of other body fluids comprise the most widely used diagnostic and therapeutic tools employed today, with billions of tests carried out annually, primarily by automated instruments and apparati. Currently utilized methods for evaluating body fluids which utilize automated instruments require relatively large amounts of the body fluid, such as a blood sample, in order to perform the evaluation. This requirement is problematic in cases where a large volume of the body fluid is not available for diagnosis, such as in newborns. The problem becomes even more acute in view of the fact that, in most cases, in order for a physician to diagnose a certain condition in an individual, it is necessary to carry out multiple tests, each requiring a separate sample. For example, in order to diagnose the presence of inflammation or to assess the intensity of an inflammation at the present time, it is necessary to send for different tests that are performed in different laboratories with different instruments, reagents and skilled personnel.

An additional drawback to presently used methods arises from the fact that due to the relatively large amount of body fluid required for each diagnostic test, some patients do not have a sufficient quantity of the relevant fluid to repeat the test over short intervals of time. This drawback imposes severe limitations on diagnosis using such methods since test repetition is often required for monitoring the progression of a clinical condition over time.

Accordingly, testing methods have been developed which require a relatively smaller amount of a fluid sample. Such tests often employ visual examination of the fluid and therefore require that the sample be held and displayed within an optical testing apparatus in a specific manner, depending upon the test involved.

Some tests require only a very small specimen. The typical manner of holding and displaying such a small specimen is to deposit a drop of the fluid sample on a horizontally held flat glass slide and allow it to spread according to its viscosity and surface tension, with or without a cover plate. The slide is then inserted into a microscope stage or other such slide handler for examination or testing by a microscope or other optical device. For such tests that require only a tiny drop of specimen, it is relatively easy to correctly deposit the specimen on the slide.

However, other tests require the deposit of a larger quantity of the specimen, which is often difficult to accomplish correctly. One type of test in which a larger specimen is required is the examination of the particulate components of a body fluid. Examples of such tests include examination of the adhesiveness/aggregation state of blood components. For example, a leukocyte adhesiveness/aggregation test (LAAT), based on the aggregation of white blood cells to one another, is used as a tool for diagnosing the presence of an inflammatory response. LAAT has also been proposed as a method of discriminating between bacterial infections (in which there is a high level of leukocyte aggregation) and viral infections (wherein attenuated or no leukocyte aggregation is detected). Other examples of such tests include the erythrocyte adhesiveness/aggregation test (EAAT) and the platelet adhesiveness/aggregation test (PAAT) which may be used to assess an atherosclerosis risk.

The above cited tests and other tests which analyze body fluid components require the generation of a profile of the particulate components of the body fluid. A common method of generating such a profile is by causing a quantity of the body fluid to flow in a controlled manner on a substrate and optically reading and analyzing the resulting distribution of the particulate components on the substrate.

Two common methods of causing such a controlled flow include depositing a specimen of the body fluid on the substrate in a sufficient quantity and causing it to flow, as described in PCT/IL00/00673, by either orienting the substrate at an angle or by placing the substrate in a centrifuge, such as a clinical centrifuge. In both methods, it is critical to carry out the procedure precisely. The correct amount of the sample must be deposited in the right location on the substrate so that the specimen flows in such a way that a readable deposit is left on the surface of the substrate. Depositing too little specimen or orienting the slide at too flat an angle or revolving at too slow a centrifuge speed may result in insufficient flow and too thick a layer for optical reading and analysis. Depositing too much specimen or orienting the slide at too great an angle or revolving at too great a centrifuge speed may cause the specimen to flow off the slide and contaminate the testing apparatus, requiring the apparatus to be decontaminated before conducting a subsequent examination on a different fluid sample.

There is thus a widely recognized need for, and it would be highly advantageous to have, a specimen carrier which allows a biological sample to be examined and analyzed as required in an apparatus and which prevents the sample from contaminating the apparatus.

### SUMMARY OF THE INVENTION

Accordingly, it is an object of the present invention to provide a fluid sample carrier that is easy and reliable to use and that holds and displays a fluid biological sample in such a manner that its particulate components may be examined and analyzed.

It is a further object of the present invention to provide a fluid sample carrier that retains a fluid biological sample within a testing apparatus without contaminating the testing apparatus.

According to the present invention there is provided a fluid sample carrier comprising at least one lane occupying a length, and a portion of a width, of a surface of the carrier, the lane being sized and configured such that a fluid sample deposited therein would flow within the lane upon exertion of a directional force upon the fluid sample. According to features in the described preferred embodiments, the carrier comprises multiple lanes usable for carrying out repetitive procedures.

According to features in the described preferred embodiments, the lane forms a groove in the surface of the carrier.

According to features in the described preferred embodiments, a cross section of the groove is U-shaped, V-shaped, semicircular shaped or square shaped.

According to features in the described preferred embodiments, a surface of the lane is configured so as to minimize capillary interactions between a surface of the lane and the fluid sample.

According to features in the described preferred embodiments, a surface of the lane is configured so as to minimize capillary interactions between a boundary of a surface of the lane and the fluid sample.

According to features in the described preferred embodiments, the fluid sample is a fluid biological sample and a surface of the lane is coated with a specific molecule or other biological material capable of binding a specific component present in the fluid biological sample.

According to features in the described preferred embodiments, a surface of the lane is coated with a substance selected for preventing or reducing capillary interactions between the surface of the lane and the fluid biological sample.

According to features in the described preferred embodiments, a surface of the lane is coated with a substance selected for preventing or reducing capillary interactions between the boundaries of the at least one lane or the surface thereof and the fluid biological sample.

According to features in the described preferred embodiments, a surface of the lane is coated with at least two specific types of molecules or biological materials, each type being capable of binding a specific component present in the fluid biological sample.

According to features in the described preferred embodiments, a first end of the lane is formed with a reservoir for accepting the biological sample.

According to features in the described preferred embodiments, a first end of the lane includes a marking for placement of the biological sample.

According to features in the described preferred embodiments, a second end of the lane contains an absorptive material for absorbing the biological sample.

According to features in the described preferred embodiments, a second end of the lane is configured for collecting the fluid biological sample following the flowing thereof.

According to features in the described preferred embodiments, a surface of the lane is textured.

According to features in the described preferred embodiments, a surface of the lane is formed with a grid pattern.

According to features in the described preferred embodiments, the carrier further comprises a cover designed and configured for covering at least a portion of the surface of the carrier.

According to features in the described preferred embodiments, at least a portion of the cover is composed of a transparent material.

According to features in the described preferred embodiments, the cover is designed for preventing contamination of a fluid biological sample deposited on the carrier.

According to features in the described preferred embodiments, the carrier is formed of a transparent material.

According to features in the described preferred embodiments, the carrier is designed and configured for placement in a microscope stage.

According to features in the described preferred embodiments, the carrier is designed and configure to be manipulated by a slide handling device.

According to features in the described preferred embodiments, the carrier is designed and configured to prevent contamination of an apparatus holding the carrier.

The present invention successfully addresses the shortcomings of the presently known configurations by providing a sample carrier that is easy and reliable to use, that displays a fluid biological sample in such a manner that its particulate components may be examined and analyzed, and that holds the sample within a testing apparatus without contaminating the apparatus or sample.

### BRIEF DESCRIPTION OF THE DRAWINGS

With specific reference now to the drawings in detail, it is stressed that the particulars shown are by way of example and for the purposes of illustrative discussion of the preferred embodiment of the present invention only, and are presented in the cause of providing what is believed to be the most useful and readily understood description of the principles and conceptual aspects of the invention. In this regard, no attempt is made to show structural details of the invention in more detail that is necessary for a fundamental understanding of the invention, the description taken with the drawings making apparent to those skilled in the art how the several forms of the invention may be embodied in practice.

In the drawings:

FIG. 1 illustrates one configuration of the biological sample carrier of the present invention;

FIGS. 2a-d illustrate various cross sectional configurations of a lane formed within the surface of the biological sample carrier of FIG. 1;

FIG. 3 illustrates another configuration of the biological sample carrier of the present invention;

FIG. 4 illustrates yet another configuration of the biological sample carrier of the present invention;

FIG. 5 illustrates still another configuration of the biological sample carrier of the present invention;

FIG. 6 illustrates yet still another configuration of the biological sample carrier of the present invention;

FIG. 7 illustrates the biological sample carrier of FIG. 1 fitted with a cover for preventing sample or apparatus contamination; and

FIG. 8 illustrates yet another configuration of the biological sample carrier of the present invention.

### DESCRIPTION OF THE PREFERRED EMBODIMENTS

The present invention is of a fluid sample carrier which greatly facilitates sample preparation and processing thus enabling high throughput analysis of fluid samples such as a biological fluid sample while preventing sample and/or apparatus contamination

The principles and operation of a biological sample carrier according to the present invention may be better understood with reference to the drawings and accompanying descriptions.

Before explaining at least one embodiment of the invention in detail, it is to be understood that the invention is not limited in this application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

Currently utilized methods for evaluating fluid biological samples which use automated instruments require relatively large amounts of body fluid such as a blood sample in order to perform an accurate diagnosis. This drawback imposes severe limitations since test repetition is often required for accurate diagnosis or for monitoring the progression of a clinical condition over time. Accordingly, methods have been developed for analyzing minimal volumes of fluid biological samples which are capable of enabling accurate diagnoses.

The present invention is of a sample carrier which enables analysis of the particulate components of, for example, a body fluid sample (e.g., blood), thus enabling generation of a profile of the particulate components of the body fluid sample, which profile can be utilized to detect and diagnose a number of clinical conditions such as an inflammatory response or condition, atherosclerosis and others.

As used herein, the term “profile” refers to an image of a body fluid sample which is representative of such a sample and which provides an indication of an individual's clinical condition.

As used herein, the phrase “body fluid” refers to a fluid biological sample obtained from a tested individual. Preferably, the sample is a blood sample obtained by standard techniques such as a finger prick or venous drawing. Other body fluids utilizable by the present invention are urine, saliva, lymph fluid, milk, cerebrospinal fluid, etc. It is understood that the term “body fluid” refers to samples taken directly from the tested individual as well as samples that have been processed, altered or treated in some manner prior to testing, such as, for example, samples treated to extract polynucleotides.

As used herein, the phrase “particulate components” refers to cellular and non cellular components of a body fluid, including, but not limited to, white blood cells, red blood cells, platelets, bacteria, hemoglobin, plasma proteins and the like.

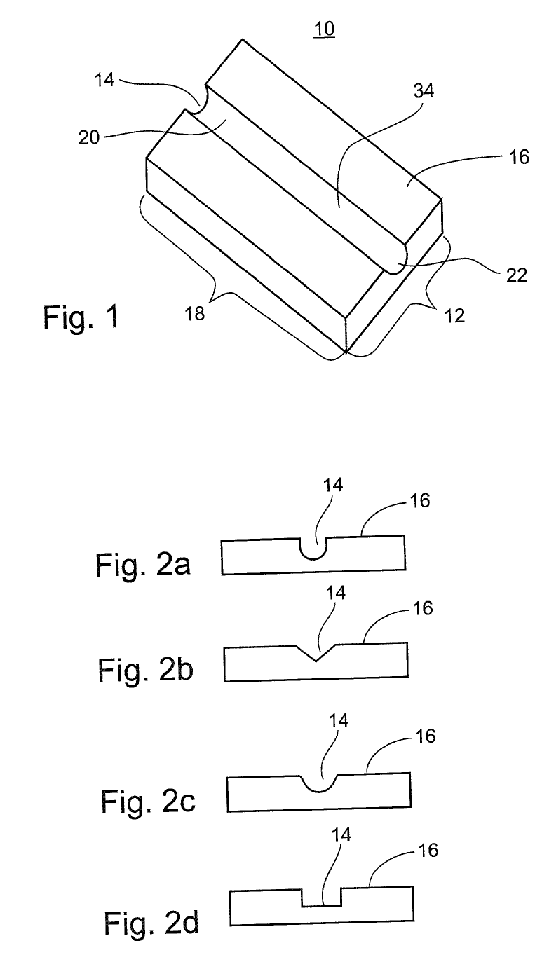

Thus, according to the present invention, and as illustrated in FIGS. 1-7, there is provided a fluid sample carrier, which is referred to herein as carrier 10.

Carrier 10 can be formed of any material suitable for supporting and displaying a fluid sample. Examples include, glass, polymers, metal and any combinations thereof. Preferably, carrier 10 is fabricated from a transparent material such as plastic or glass. The dimensions of carrier 10 are selected according to the fluid sample to be examined and/or the apparatus used for sample analysis. For example, when used along with a microscope (e.g., light or fluorescent microscope), carrier 10 is selected of dimensions suitable for positioning within a microscope stage, e.g. a width of 20-50 mm and a length of 60-120 mm.

Carrier 10 includes one or more lanes 14 formed within a surface 16 of carrier 10. Lane 14 occupies a length 18 and a portion of the width 12 of carrier 10. Lane 14 serves for holding the fluid sample and is sized and configured such that a fluid sample deposited at one end 20 of lane 14 flows to a second end 22 of lane 14 upon application of a directional force thereupon. Such flow of the fluid sample creates a thin film of the fluid sample within lane 14 and as such greatly facilitates analysis of the particulate components contained in the fluid sample.

According to a preferred embodiment of the invention, lane 14 is formed as a groove in surface 16. Lane 14, so configured as a groove, may be fabricated by engraving/etching a channel in surface 16 of carrier 10 or by molding carrier 10 with lane 14. Lane 14 can also be fabricated by adding material to surface 16 on both sides of lane 14 so as to form “walls” on either side of lane 14 thereby defining lane 14 on surface 16.

Lane 14 may be of any cross sectional shape and depth. Examples of various cross sectional shapes of lane 14 are provided in FIGS. 2a, 2b, 2c and 2d.

FIG. 2a illustrates a U-shaped lane 14. In this cross sectional configuration, lane 14 is preferably selected of a depth ranging between 0.2-0.3 mm. Such a configuration of lane 14 is advantageous in that it enables a uniform flow of the fluid sample, thereby providing a uniformly deposited film of fluid on surface 34 of lane 14 and it prevents a possible “leakage” or sticking of part of the fluid sample under or on sharp wall edges and boundary lines. It is understood that the cross section of lane 14 may be V-shaped as shown in FIG. 2b, semicircular shaped as shown in FIG. 2c, square shaped as shown in FIG. 2d or of any other shape provided that the shape be so designed and configured to allow a fluid sample to flow therein and to leave a film deposit on surface 34. Lane 14 is also preferably designed and configured to minimize capillary interactions between lane surface 34 or the boundaries of lane 14 and the fluid sample flowing therein. In this respect, Lane 14 may also be coated with a substance selected for preventing or reducing such capillary interactions.

Lane 14 may vary in size, shape and orientation with respect to carrier 10. For example, the direction of lane 14 with respect to the length and the width of carrier 10 may vary, with lane 14 oriented transversely such that it occupies the entire width 12 and a portion of length 18, or with lane 14 oriented diagonally with respect to both length 18 and width 12. Moreover, lane 14 may be sized and shaped according to the characteristics of the body fluid sample to be tested or the requirements of the test. For example, lane 14 may be straight or curved, wide or narrow, deep or shallow, have parallel, converging or diverging edges or be irregularly shaped. It is understood that the position, orientation and shape of lane 14 on carrier 10 are all subject to modification according to the needs of the test or examination to be performed.

Moreover, lane surface 34 may be formed with a smooth or textured surface, depending upon the desired deposition of the fluid sample thereon. Lane surface 34 may, for example, have a square or diamond shaped grid pattern or be formed with shapes having slightly raised ridges designed to trap small and equal quantities of the fluid sample for the purpose of taking multiple readings for examination. It is understood that lane surface 34 may be treated and its properties modified as needed in order to effect the desired characteristics and/or behavior of the fluid deposited thereon. All such treatments and modifications to lane surface 34 are contemplated by the present invention.

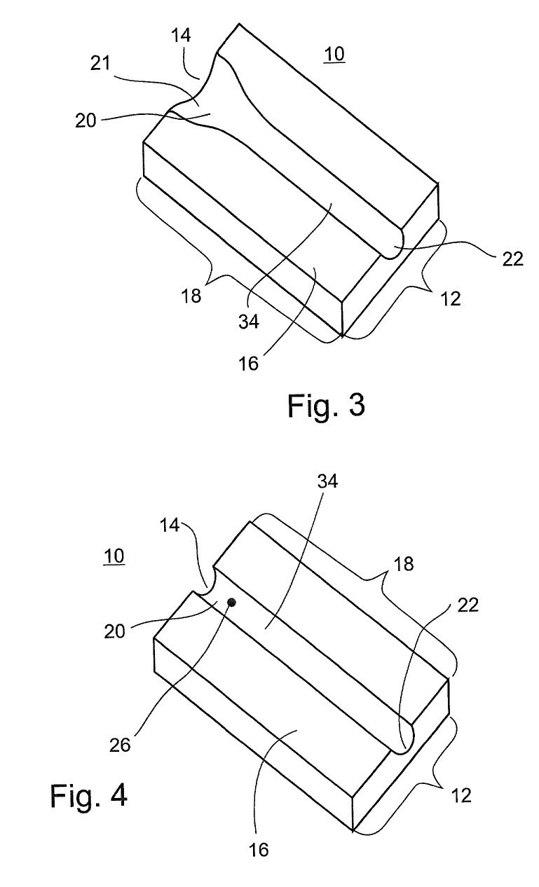

In this spirit, lane 14 may be formed according to alternate configurations as needed or desired for different applications. Reference is made to FIG. 3 which shows lane 14 formed with a widened reservoir 21, at end 20. Reservoir 21 permits the deposit of a larger amount of the fluid sample for testing should that be a requirement of the examination.

As shown in FIG. 4, lane 14 can also be formed with a marking 26, which serves to indicate the correct placement of a fluid sample to be tested.

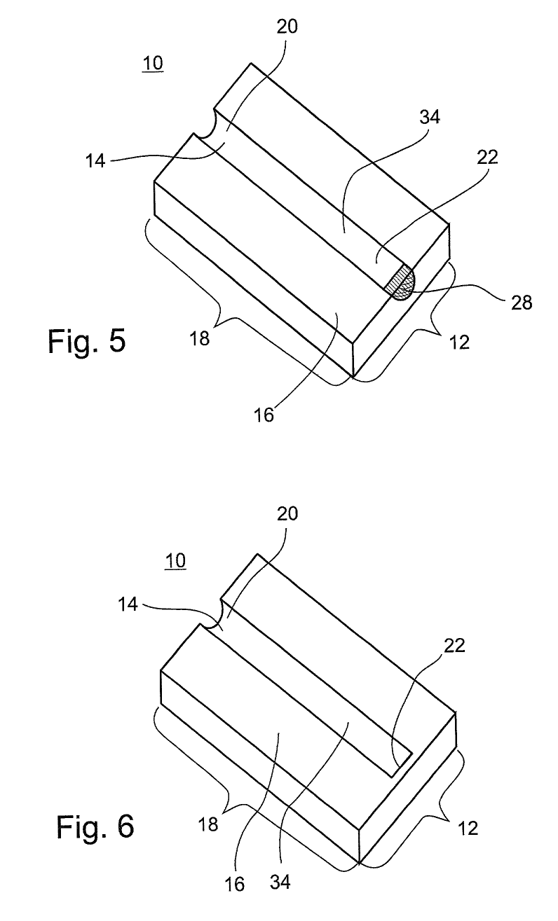

In order to prevent fluid sample from escaping carrier 10 and thus contaminating the testing apparatus, end 22 includes either a collection reservoir (not shown), a block of an absorbing material 28, which may be covered by a non-absorbing material (FIG. 5) or a physical barrier (FIG. 6).

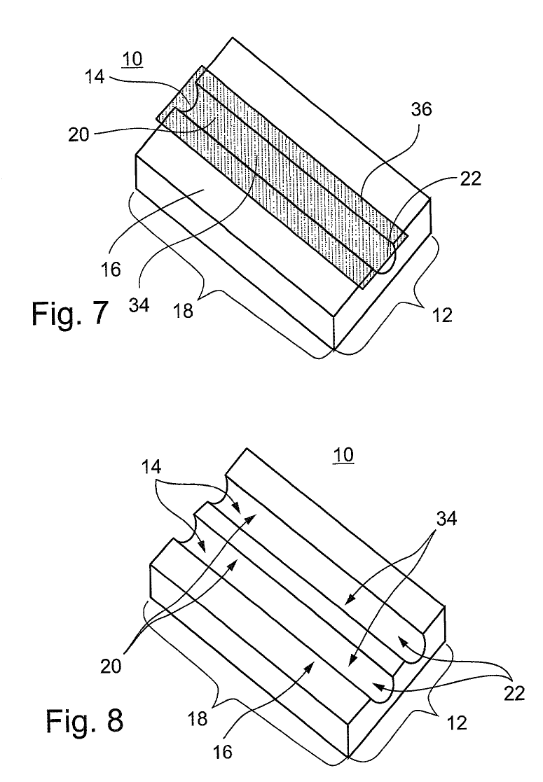

Carrier 10 may also include a cover 36 (FIG. 7), which serves to cover lane 14 thus preventing contamination of a fluid sample contained therein, or contamination of a testing apparatus by preventing escape of the fluid sample. Cover 36 is preferably designed and configured to address the contamination risk prevalent with the test being conducted. The potential for contamination may be exposure to other biological materials, inadvertent contact with laboratory instruments or the testing apparatus, or simply the entry of specks of dust or other foreign material. Cover 36 is preferably formed of a transparent material and may be so sized to cover all of lane 14 or a portion thereof and its ends.

Carrier 10 is specifically, but not exclusively, designed and configured for use in tests and examinations that require generating a profile of particulate components of a body fluid sample (e.g., blood). Such design and configuration is not meant to be a limitation on the applications for which carrier 10 is employed. It is foreseeable that carrier 10 will be useful and appropriate for many optical, chemical and other tests to which a body fluid sample, or any other fluid sample, may be subjected. Accordingly, carrier 10 may preferably be designed and configured for placement in a microscope stage, to be manipulated by a slide handling device or to adapt to any special apparatus or any special fitting or holder in an analysis device. Moreover, all similarly configured versions of carrier 10 will preferably be designed to be handled by the testing apparatus in the same way in order to provide uniformity in procedures so as to remove this element as a variable factor in comparative analyses or cumulative data. It is understood that carrier 10 may be designed so as to be compatible with any testing apparatus required for the relevant tests or examinations to be performed.

Preferably, when used for analysis of a biological fluid, such as blood, carrier 10 includes a plurality of lanes 14 as shown, for example, in FIG. 8, each being capable of carrying a biological sample. Carrier 10 with multiple lanes of the same configuration might be useful, for example, for carrying out repetitive procedures in which all samples have the same flow characteristics.

Lane surface 34 may be optionally coated with antibodies, DNA or RNA strands or probes, chemicals or other substances capable of modifying the property of surface 34. For example, it may be coated with one or more specific molecules (e.g., antibodies) or other biological materials which are capable of selectively capturing specific cell types, such as anti CD11b/CD18 antibodies which capture white blood cells or anti IIb/IIIa antibodies which capture platelets, or with dyes which connect with specific components of the sample.

Numerous testing methods and apparati can benefit from the advantages in biological sample processing and display presented by carrier 10 of the present invention.

Due to its advantages in displaying particulate components, carrier 10 of the present invention is most suitable for use with the INFLAMET™ SYSTEM, which is described at an Internet Website accessible via URL www.inflamet.com.

When used with this system, carrier 10 is preferably held in a position angled from the horizontal in order to allow controlled flow of a body fluid sample within lane 14. Alternatively, such a controlled flow may be accomplished by placing carrier 10 in a centrifuge and exerting a gentle centrifugal force (1-10 g) on the body fluid sample placed in lane 14. It will be appreciated that in the later case, carrier 10 can be designed to be placed in a specially adapted holder for use with a specific centrifuge model.

When subjected to such a force, the body fluid sample flows within lane 14 and forms a thin film coating upon surface 34 of lane 14. During this flowing movement, each particulate component of the body fluid sample adheres to lane surface 34 at a position which is dependent on the size, aggregation tendencies and the adherence properties of the component. In general, smaller aggregates or less “sticky” components tend to move a greater distance on lane surface 34 than larger aggregates or more “sticky” components. This differential distribution of the particulate components on lane surface 34 represents a profile of particulate components of the body fluid sample. The specific design and configuration of carrier 10 is devised to enhance and facilitate the controlled flow process, and to do so while protecting the testing apparatus from contamination by the tested body fluid sample.

This system and many others for which carrier 10 is compatible are commonly employed to carry out many tests, such as volumetric analyses, blood cell counts and differential analyses, aggregation and adhesion analyses, and many others. The INFLAMET™ SYSTEM is also used to characterize the profile of the particulate components of a body fluid sample according to many parameters, including, but not limited to, estimated hemoglobin concentration, approximated leukocyte count and differential, approximated platelet count, degree of leukocyte aggregation, aggregate composition, degree of leukocyte, erythrocyte and/or platelet adherence towards the surface of said substrate, degree of red cell aggregation, degree of platelet aggregation, degree of leukocyte to erythrocyte interaction, degree of erythrocyte to platelet interaction and degree of leukocyte to platelet interaction.

Carrier 10 of the present invention provides a distinct advantage over prior art slides and substrates by facilitating diagnostic tests from a minimal volume of a body fluid sample, thus making such tests available to infants and others from whom a large volume of blood and other fluids cannot be drawn.

In addition carrier 10 facilitates testing of numerous samples concomitantly (multi-lane carriers) thus enabling high throughput screening of fluid samples. Such samples may be from the same or from different patients.

Moreover, since carrier 10 serves for both sample preparation (fluid sample flow) and analysis (e.g., microscope analysis), use thereof minimizes sample handling and as such minimizes sample contamination, sample loss and the like.

Finally, the unique design and configuration of carrier 10 prevents contamination of testing apparati thus greatly reducing the likelihood of cross sample contamination and other problems associated with the contamination of diagnostic apparati.

It will be appreciated by persons skilled in the art that the present invention is not limited to what has been particularly shown and described hereinabove. Rather, the scope of the present invention is defined by the appended claims and includes both combinations and subcombinations of the various features described hereinabove as well as variations and modifications thereof which would occur to persons skilled in the art upon reading the foregoing description. Accordingly, it is intended to embrace all such alternatives, modifications and variations that fall within the spirit and broad scope of the appended claims.

What is claimed is:

1. A fluid sample carrier comprising at least one lane occupying a length, and a portion of a width, of a surface of the carrier, said at least one lane being sized and configured such that a fluid sample deposited in said at least one lane would flow within said at least one lane upon exertion of a directional force upon said fluid sample.

2. The carrier of claim 1, wherein said at least one lane forms a groove in said surface of the carrier.

3. The carrier of claim 2, wherein a cross section of said groove is U-shaped, V-shaped, semicircular shaped or square shaped.

4. The carrier of claim 1, wherein a surface of said lane is configured so as to minimize capillary interactions between a surface of said at least one lane and said fluid sample.

5. The carrier of claim 1, wherein a surface of said lane is configured so as to minimize capillary interactions between a boundary of a surface of said at least one lane and said fluid sample.

6. The carrier of claim 1, wherein said fluid sample is a fluid biological sample and whereas a surface of said at least one lane is coated with a specific molecule or other biological material capable of binding a specific component present in said fluid biological sample.

7. The carrier of claim 1, wherein a surface of said at least one lane is coated with substance selected for preventing or reducing capillary interactions between said surface of said at least one lane and said fluid biological sample.

8. The carrier of claim 1, wherein a surface of said at least one lane is coated with substance selected for preventing or reducing capillary interactions between a boundary of said at least one lane or a surface thereof and said fluid biological sample.

9. The carrier of claim 1, wherein said fluid sample is a fluid biological sample and whereas a surface of said at least one lane is coated with at least two specific types of molecules or other biological materials, each type being capable of binding a specific component present in said fluid biological sample.

10. The carrier of claim 1, wherein a first end of said at least one lane is formed with a reservoir for accepting said biological sample.

11. The carrier of claim 1, wherein a first end of said at least one lane includes a marking for placement of said biological sample.

12. The carrier of claim 1, wherein a second end of said at least one lane contains an absorptive material for absorbing said biological sample.

13. The carrier of claim 1, wherein a second end of said at least one is configured for collecting said fluid biological sample following flowing thereof.

14. The carrier of claim 1, wherein a surface of said at least one lane is textured.

15. The carrier of claim 1, wherein a surface of said at least one lane is formed with a grid pattern.

16. The carrier of claim 1, further comprising a cover designed and configured for covering at least a portion of said surface of the carrier.

17. The carrier of claim 16, wherein at least a portion of said cover is composed of a transparent material.

18. The carrier of claim 16, wherein said cover is designed for preventing contamination of a fluid biological sample deposited on the carrier.

19. The carrier of claim 1, wherein the carrier is formed of a transparent material.

20. The carrier of claim 1, wherein the carrier is designed and configured for placement in a microscope stage.

21. The carrier of claim 1, wherein the carrier is designed and configured to be manipulated by a slide handling device.

22. The carrier of claim 1, wherein the carrier is designed and configured to prevent contamination of an apparatus holding the carrier.
